(19) United States
(12) Patent Application Publication
(10) Pub. No.: US 2025/0177752 A1
(43) Pub. Date: Jun. 5, 2025

(54) INTRA-LUMINAL MULTI-SITE BIO-STIMULATION, SENSING & MAPPING DEVICE WITH AUTO FEEDBACK

(71) Applicant: Yale University, New Haven, CT (US)

(72) Inventors: Pramod Bonde, New Haven, CT (US); Rigu Gupta, New Haven, CT (US)

(21) Appl. No.: 18/840,419

(22) PCT Filed: Feb. 22, 2023
(86) PCT No.: PCT/US2023/062988

§ 371 Date: Aug. 21, 2024

Related U.S. Application Data
(60) Provisional application No. 63/312,497, filed on Feb. 22, 2022.

(51) Int. Cl. A61N 1/36; A61B 5/0538; A61N 1/05

(52) U.S. Cl. CPC A61N 1/36139; A61B 5/0538; A61N 1/0509; A61N 1/36007

(57) ABSTRACT

A stimulation catheter is disclosed. The catheter has an elongate flexible member and multiple resonator coils configured between its proximal and distal end. A cathode is connected to each of the resonator coils. Multiple anodes are each connected to and corresponding with one of the resonator coils. Multiple stimulation elements are each connected to and corresponding with one of the resonator coils. A control unit is coupled to the cathode and the anodes, and is configured to generate a stimulation signal based on feedback signals generated from the resonator coils. A stimulation catheter system is also disclosed.

### CROSS-REFERENCE TO RELATED APPLICATIONS

This application claims priority to U.S. provisional application No. 63/312,497 filed on Feb. 22, 2022 incorporated herein by reference in its entirety.

### BACKGROUND OF THE INVENTION

Impaired swallowing is observed in many patients who have suffered a stroke or a brain injury. Animal studies have shown that the central influence of the nervous system is not necessary to initiate a swallowing reflex when direct local electrical stimulation is performed. The swallowing mechanism is important for clearance of oral secretions, pharyngeal secretions as well as secretions coming from the sinuses. When patients are put on artificial respiration, they usually have a breathing tube that is inserted down their mouth or nose into the trachea. To tolerate the presence of the breathing tube and to facilitate successful patient breathing with the ventilator, sedatives and other paralytic drugs are given to the patient which inhibit the central pathway for swallowing. A normal human being produces 700 to 1200 CC of salivary secretions and additional secretions from the sinuses. These secretions tend to collect in the posterior pharynx and these patients are usually recumbent, so these secretions are at risk of entering the breathing system and causing pneumonia in the lung.

In addition, patients who have a breathing tube and are on ventilators have impaired clearance of gastric juices which typically amounts to 1-1.5 liters per day. The clearance of the stomach contents is dependent on the adequate functioning of duodenum and jejunum. The relaxation of the upper and the lower food pipe valve causes these stomach contents to freely regurgitate in the trachea and subsequently in the lungs causing aspiration or pneumonia, also known as ventilator associated pneumonia. Simply stimulating the food pipe is inadequate for achieving coordinated emptying of the oral secretions, gastric secretions, duodenal contents, and Jejunal clearance.

Stimulation of nerves such as the superior laryngeal nerve and branches of the glossopharyngeal nerve are important in initiating the swallowing. Subsequent relaxation of the upper esophagus sphincter is initiated with this stimulation, creating a secondary peristaltic wave within the esophagus which in turn relaxes the lower sphincter, thus clearing the secretions and pushing them into the stomach. Clearance of the stomach is more complicated with the antral portion of the stomach playing an important role in the emptying of the stomach. However, emptying of the stomach is dependent on jejunal clearance and hence stimulation of the jejunum in turn will lead to increased stomach clearance.

Thus, what is needed in the art is a device that can detect physiological feedback along an anatomical lumen, while having the ability to generate stimulation signals coinciding with the proximity of target stimulation point along the anatomical lumen.

### SUMMARY OF THE INVENTION

In one embodiment, a stimulation catheter includes an elongate flexible member having a proximal end, a distal end, and resonator coils configured therebetween; a cathode connected to each of the resonator coils; anodes each connected to and corresponding with one of the resonator coils; stimulation elements each connected to and corresponding with one of the resonator coils; and a control unit coupled to the cathode and the anodes, wherein the control unit is configured to generate a stimulation signal based on feedback signals generated from the resonator coils. In one embodiment, the control unit comprises a controller communicatively coupled to each of the plurality of anodes and each of the plurality of stimulation elements. In one embodiment, the controller is configured to generate a sequence of stimulation signals for the stimulation elements. In one embodiment, the sequence is based on detecting a predetermined threshold sequence of physiological feedback from the two or more of the resonator coils. In one embodiment, the sequence activates the plurality of stimulation elements in an order moving in a distal direction. In one embodiment, the sequence activates the stimulation elements in an order moving in a proximal direction. In one embodiment, the sequence activates combinations of two or more of the plurality of stimulation elements at different times. In one embodiment, the control unit includes a multiplexer connected to each of the anodes. In one embodiment, the feedback signals includes at least one of a resistance, admittance, impedance, reactance, susceptance, capacitance, or conductance between inductively coupled electrode measurement signal. In one embodiment, at least one of the feedback signals includes a zero-value measurement. In one embodiment, the resonator coils are spaced apart by a uniform distance. In one embodiment, the resonator coils are spaced apart by different distances. In one embodiment, the resonator coils are configured on an outer surface of the elongate flexible member. In one embodiment, each of the resonator coils wraps around the outer surface at least one time in a spiral formation. In one embodiment, each of the resonator coils wraps around an outer surface at least two times time in a spiral formation. In one embodiment, the elongate flexible member includes a lumen extending from the proximal end to a distal tip. In one embodiment, the elongate flexible member includes a medical grade polymer. In one embodiment, the medical grade polymer is polyamide. In one embodiment, at least one of the stimulation elements is a capacitor. In one embodiment, the capacitor is a surface mount device capacitor configured on a surface of at least one of the plurality of resonator coils. In one embodiment, a stimulation catheter system includes an elongate flexible sheath having a sheath lumen connected to a distal sheath opening, and multiple sheath stimulation elements surrounding the sheath lumen. In one embodiment, a stimulation catheter system includes the catheter and an elongate flexible internal body having a distal tip sensor. In one embodiment, the distal tip sensor is a pH sensor. In one embodiment, the elongate flexible internal body comprises a plurality of body stimulation elements. In one embodiment, the control unit is configured to predict a media type based on a detected resistance. In one embodiment, the control unit is configured to amplify detection by spatially tuning adjacent electrodes. In one embodiment, the device includes embedded pressure sensors on an external aspect of the hollow longitudinal tube configured to detect pressure at different levels of the esophagus, stomach, intestine or urinary bladder corresponding to a site of use. In one embodiment, the device includes an obturator within the outer sheath, where the obturator has a distal end viewing camera. In one embodiment, the device includes an obturator tip can having a pH sensor, optical fluorescence sensor and a pressure sensor, wherein the sheath is configured to be a carrier for a tube like structure within its lumen after the removal of the obturator.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is a diagram of a stimulation device according to one embodiment, and FIG. 1B is a diagram of stimulation device components according to one embodiment.

FIG. 2 is a diagram of stimulation device components and circuits according to one embodiment.

FIG. 3A is a sensing block diagram and an op-amp circuit diagram according to one embodiment, and FIG. 3B is a pacing block diagram, a circuit diagram of a pace electrode oscillator and a block diagram of a pulse output circuit according to one embodiment.

FIG. 4A is a diagram of an equivalent circuit model of a multi-coil resonator system according to one embodiment, and FIG. 4B is a table of coil parameters and their formulas according to one embodiment.

FIG. 5A is a diagram of return loss and impedance matching according to one embodiment, FIG. 5B is a diagram of an output display according to one embodiment, and FIG. 5C is a table of impedance and admittance parameters according to one embodiment.

FIG. 6 is a perspective view of a stimulation sheath and internal body according to one embodiment.

FIGS. 7A-7L show images of experimental data. FIG. 7A is an image of a gross appearance of reflux leading to increased oral secretions soiling the mouth and nasal area, FIG. 7B is an image of gastric distention with retained gastric contents, FIG. 7C is an image of the gross appearance of aspiration pneumonia in lungs with increased weight, edema and infection, FIG. 7D is an image of micro abscesses within the lung parenchyma, FIG. 6E is an image of the gross appearance of esophagus with a distended lower portion, FIG. 7F is an image of a normal esophageal lumen appearance, FIGS. 7G and 7H are images of reflux injury of the esophagus, FIG. 7I is an image of a normal esophagus (microscopic section), FIG. 7J is an image of a mild reflux injury, FIG. 7K is an image of a severe esophageal injury due to reflux, and FIG. 7L shows images of lung damage from reflux.

FIG. 8A is a diagram of experimental data showing how the electrical activity of the heart compares to the electrical activity of the stomach, according to one embodiment. FIG. 8B is a chart of resistance measurements from a stimulation device according to one embodiment. FIG. 8C is a graph of experimental results that demonstrate resistance is different in different medium according to one embodiment.

FIG. 8D shows an experimental setup for demonstrating varying the RF signal and that a different frequency of electrical signal can be implemented to increase or decrease sensitivity of the media of interest according to one embodiment. FIG. 8E shows a graph of experimental results, demonstrating that it is possible to amplify the signal by spatially tuning two adjacent electrodes according to one embodiment.

FIGS. 8F-8I are experimental data based on different types of catheters, demonstrating the adaptability within various types of catheters. FIGS. 8J-8L show experimental data showing a difference of resistance in air vs. saline. FIGS. 8M and 8N data for pacing the gastrointestinal tract according to one embodiment.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in sensing and stimulation devices. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a sensing and stimulation device and system.

Embodiments of the device can detect physiological feedback along an anatomical lumen as a basis for generating stimulation signals coinciding with the proximity of target stimulation point along the anatomical lumen. Embodiments of the device can be utilized for several different treatments in various parts of the body. For example, embodiments of the device can be used to restore bladder emptying, bladder function, improving bladder neck tone, and for treating urinary incontinence when used intravesically (urinary bladder and intra virginal). In one embodiment, a rectal application is implemented for treating constipation and pelvic floor exercises in recumbent patient who are chronically recumbent with loss of rectal tone and resultant absence of defecation reflex. In one embodiment, an external ear application is implemented to treat tinnitus, trigeminal neuralgia and TM joint pain. In one embodiment, oral, pharyngeal and laryngeal application is implemented to treat impaired swallowing and dysphagia. The obturator can be modified to be an end viewing telescope, camera or a sensor for pH, pressure or able to induce a EM force field that is detectable by the electrodes mounted on the outer sleeve of the tube. The electrode shapes may be for example ring, longitudinal, crown or spiral shaped. The controller is able to give fixed output of electrical stimulus with for example square, triangular, or sinusoidal waves with varying voltage, amperage, time duration and amplitude.

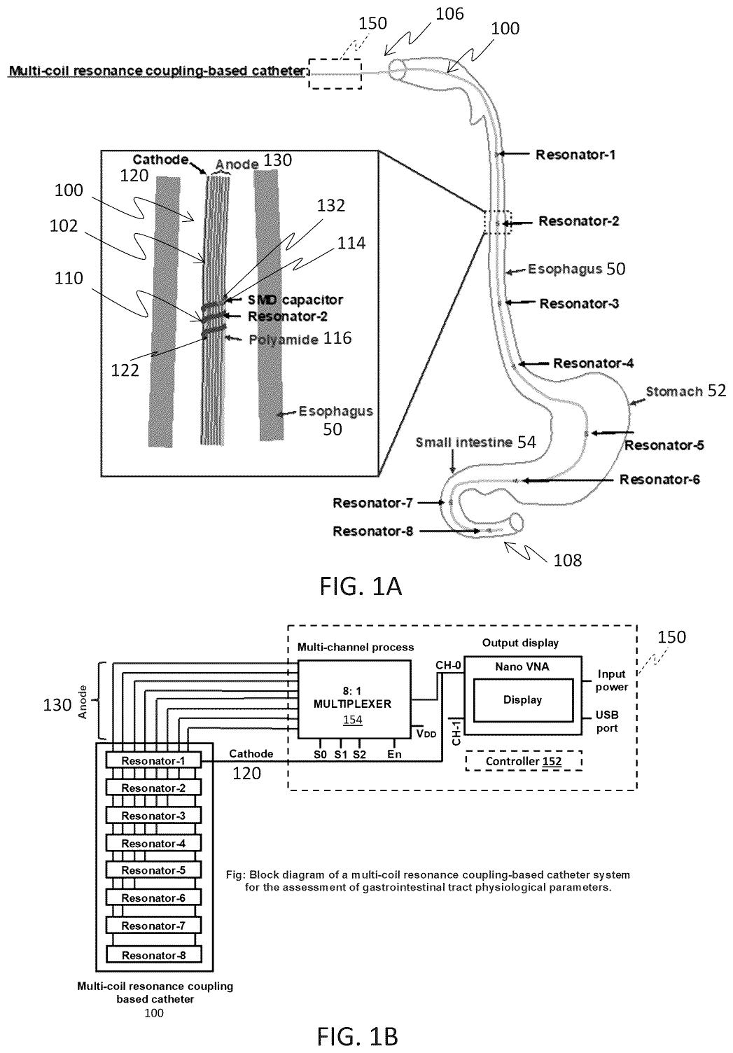

With reference now to FIGS. 1A and 1B, according to one embodiment, a stimulation catheter 100 includes an elongate flexible member 102 having a proximal end 106, a distal end 108, and a plurality of resonator coils 110 configured therebetween. The elongate flexible member 102 is configured for insertion into an anatomical lumen such as the esophagus 50, stomach 52 and small intestine 54 depicted in FIG. 1A. The elongate flexible member 102 can be advanced to a target position of interest. A cathode is 120 connected to each of the plurality of resonator coils 110 by a conductive lead at a cathode connection point 122, and multiple conductive least for anodes 130 are each connected to and corresponding with one of the resonator coils 110 at an anode connection point 132. Stimulation elements 114 such as surface mount device (SMD) capacitors are connected to and correspond with one of the resonator coils 110. A control unit 150 is coupled to the proximal end of the stimulation catheter, which can include the controller 152, multiplexer 154 and a user interface which can for example include user input and output components such as a display. The multiplexer can for example be an 8:1 multiplexer for embodiments that utilize 8 resonator coils such that the anode lead associated with each resonator coil provides an input to the multiplexer. The control unit 150 is configured to generate a stimulation signal for one or more on the stimulation elements 114 based on the feedback signals generated from the resonator coils 110. The controller can be communicatively coupled to each of the anodes and stimulation elements.

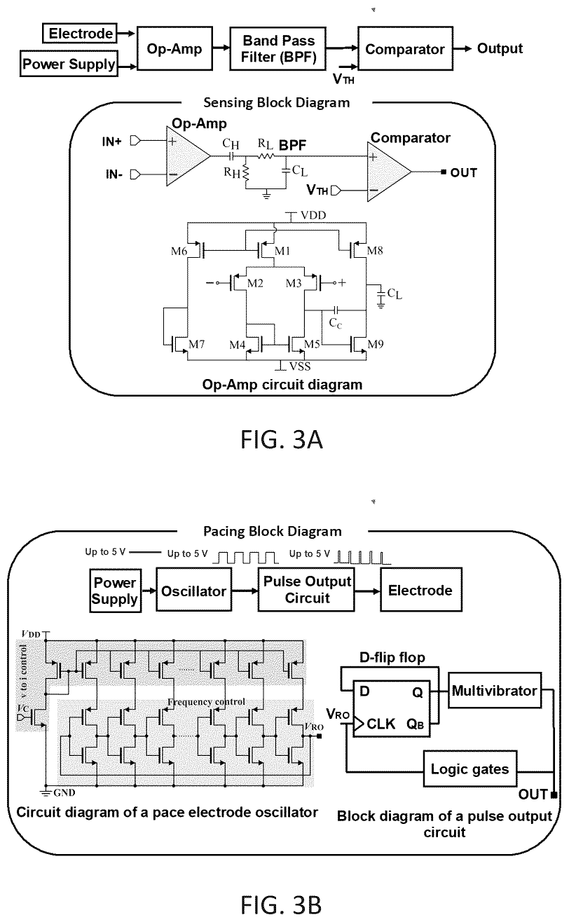

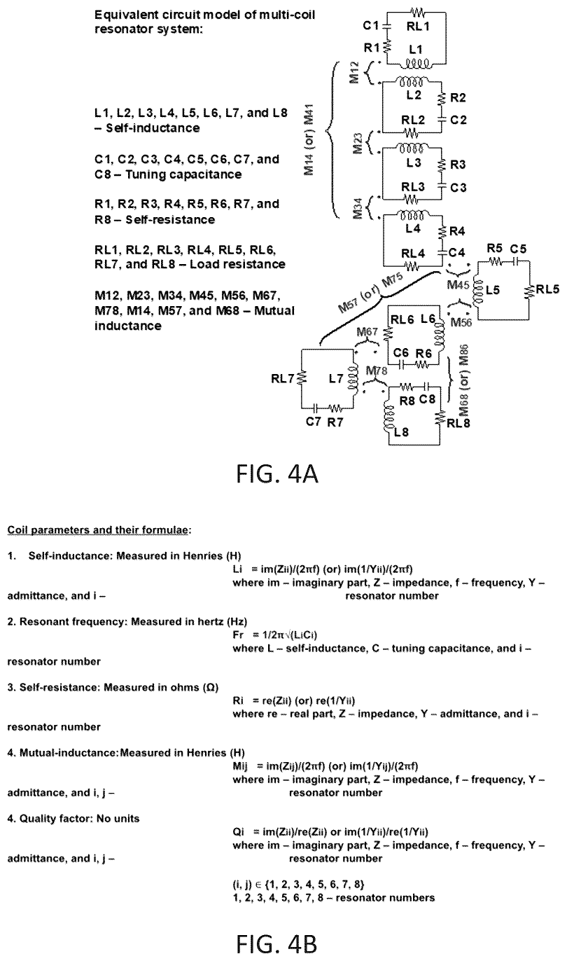

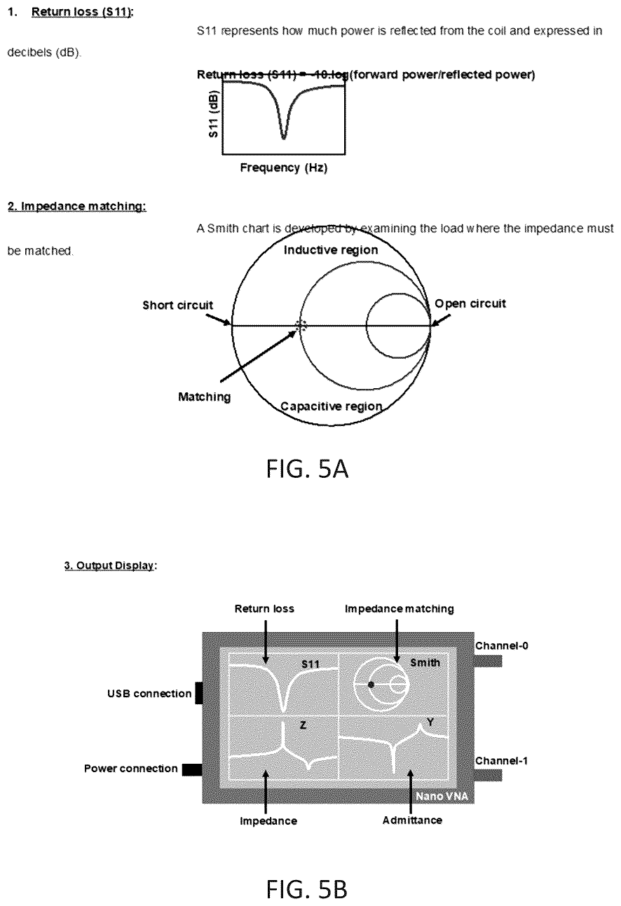

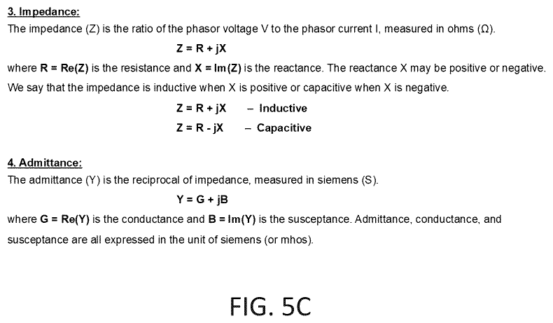

The controller can be programmed to selectively generate a sequence of stimulation signals the stimulation elements based on detecting a predetermined threshold sequence of physiological feedback from the resonator coils. A sensing block diagram and a pacing block diagram are shown according to one embodiment in FIGS. 3A and 3B. The feedback signal can for example be a zero-value measurement, a threshold positive-value measurement, or at least one of a resistance, admittance, impedance, reactance, susceptance, capacitance, or conductance between inductively coupled electrode measurement signal measurement. An equivalent circuit model for a multi-coil resonator system is shown according to one embodiment in FIG. 4A. The resonator coils can have various configurations on the flexible member, such as spaced apart by a uniform distance or spaced apart by different distances (including increased distancing, decreased distancing, or some combination or other geometric arrangement). The elongate flexible member can include a lumen extending between the proximal end and the distal tip. The lumen can be used for example to aspirate, deliver a treatment, or for housing a guidewire or some other device during placement, treatment or removal. In one embodiment, the elongate flexible member is made from a medical grade polymer, such as polyamide.

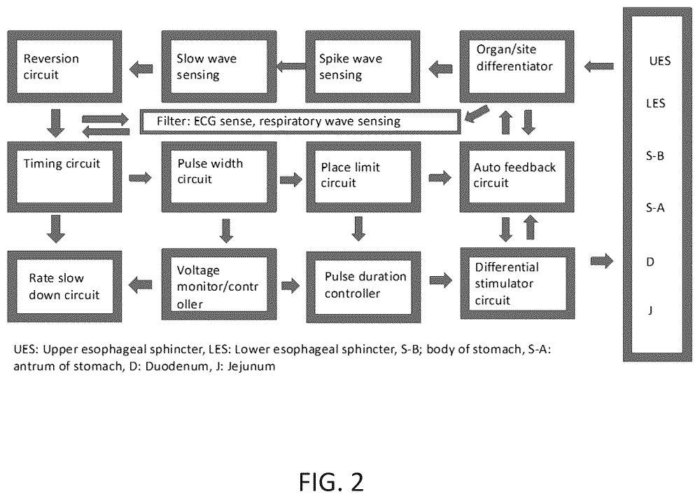

With reference now to FIG. 2, a diagram depicting a feedback and stimulation configuration is shown according to one embodiment. Embodiments of the device can detect physiological feedback sequences as a basis for generating stimulation signals in a sequence coinciding with the proximity of stimulation elements to target stimulation zones along an anatomical lumen. The control unit coupled to a proximal end of the device can include various circuits and modules for reading feedback and implementing the proper stimulation array. For example, a timing circuit can be configured to determine the basic timing rate of the pulse generator. It may consist of an RC network, a reference voltage source and a comparator. A pulse width circuit can be configured to determine the stimulating pulse duration. It is triggered by the output from the timing circuit. The pulse width circuit is also an RC circuit as the timing circuit. The output of the pulse width circuit is fed into the pace limiting circuit. The function of the pace limiting circuit is to limit the pacing rate. The maximum pacing rate is usually selected as per the target organ. The pace limit circuit limits the pacing rate by disabling the comparator for a preset interval of time. The stimulator circuit, which may also be referred to as the output circuit, provides the proper input pulse to stimulate the organ, nerve or region of interest. The refractory circuit provides a period of time following an output pulse or sensed R-wave. During this time the amplifier will not respond to outside signals. The wave sensing circuit is configured to detect or sense a spontaneous slow and spike wave (and intrinsic R wave from ECG) and to reset the oscillator when the pulse is not needed. The reversion circuit (which may also be referred to as a return circuit) allows the amplifier to detect a spontaneous slow and spike wave. In the absence of a spike wave, this circuit again allows the oscillator to generate pulses at its preset rate. This circuit is referred to as a reversion or return circuit since it allows a return of the oscillator to its active state. The voltage monitor and controller circuit continuously monitors the battery voltage. As the pacing rate is depending on the efficiency of the battery, it is monitored regularly. If the battery voltage is decreased, it triggers the energy compensation and pulse duration controller circuit. An alarm will show low battery to warn and request a charge to a fresh battery. If the battery voltage is decreased, the energy compensation circuit (or pulse duration controller circuit) increases the pulse duration so that the pulses delivered to the patient are not affected by the battery charge loss. The rate slow down circuit is a special circuit that slows down the simulation rate during certain conditions such as extreme battery depletion. This circuit shows down the rate by limiting the current to the basic timing network.

Accordingly, in one embodiment, the device provides a closed loop, autofeedback, multi-site, multi-organ, intra luminal electrical pacing and sensing system and device. This is controlled by a microcontroller, initiated by a function generator, and consists of electronics which detects the resistance, admittance, impedance, reactance, susceptance, capacitance, and conductance between inductively coupled electrode regimen. The inductively coupled regimen of the electrodes provides information between the electrodes of admittance, inductance, impedance, capacitance and susceptance which characterizes the intra luminal contents and motion. Interaction, physiological feedback and relationships between the electrodes can be configured to provide size, position and empty or filled status of the hollow organ. The device records the response to bio stimulation and conveys the information in real time. A graphic display can be utilized to allow more information in real time, which can be transmitted over to any device via Bluetooth, wifi or radio signals. In one embodiment, the electrodes are specially designed with a geometry to increase the contact area and surface area.

Embodiments of the device take advantage of physiological observations that were gathered in part during device development and experimental activity as explained in further detail below. The main purpose of the food pipe is to push the swallowed food or fluid towards the stomach. The esophagus has two valves, one is at the top where the voice box is and one at the bottom where it enters the stomach. The purpose of these two valves is to prevent entry of the swallowed food into the voice box and contaminating the lungs, and the purpose of the lower valves is to prevent the contents of the stomach from coming up into the food pipe also called esophagus. Since the stomach contents are extremely acidic, any regurgitation or reflux of these contents into the food pipe and the lungs can cause severe damage. Proper forward squeezing of the contents within the stomach is important. The stomach after mixing the food and storing the food empties into the duodenum. That duodenum contains alkaline environment which neutralizes the acid from the stomach. The contents from the duodenum are then delivered to the jejunum which is a part of the small intestine.

Esophageal peristalsis, that is, coordinated squeezing of the esophageal muscle, is a complex mechanism. The process is started by the swallowing of food. The beginning of this phase is voluntary when food is swallowed, then mixed with saliva to form a bolus of the food and then pushed by the tongue to the posterior pharynx. The next phase is involuntary, which involves a sequential squeezing of the muscles of the upper head and neck. The food bolus is then pushed forward by the esophagus and by the pharyngeal muscles. Simultaneously, movement of the muscles of the palate and elevation of the larynx is initiated so that the food does not enter the nasal cavity or the larynx. When the deglutition reflex is initiated, the upper esophageal valve also known as upper esophageal sphincter gets activated and relaxes to allow the food bolus to pass into the body of the esophagus. The sequential contraction of the circular smooth muscles initiates the peristaltic activity. The speed of this activity is about 3 centimeter per second in the upper part of the esophagus and five centimeter per second in the middle part of the esophagus, and then it becomes 2.5 centimeter per second in the last part of the esophagus. This peristaltic activity is reflected in the pressure changes in the tubular esophagus. The highest pressures are mean of 54 plus minus 8 millimeter of mercury in the upper part, 35+/−5 millimeter of mercury in the middle part and 70+/−12 millimeter of mercury in the lowest part of the esophagus. The peristaltic activity of the esophagus is affected by factors such as the food bolus size, with a larger bolus causing more stronger peristaltic contractions while a smaller bolus leads to slower peristaltic contraction. Temperatures such as warmer boluses enhances the peristaltic activity while colder boluses have a inhibit reaction on the peristaltic process.

The striated muscle fibers of the esophagus are controlled by the excitatory nerve activity from the lower motor neurons, and this is affected through the nucleus ambiguous and nucleus retro facials via the vagus nerve. Inhibition or destruction of the vagus nerve bilaterally in the cervical region can eliminate the primary peristaltic wave. However secondary peristalsis can happen independently of the central nervous system control and the secondary peristaltic waves can be independently generated. Isolated smooth muscle strips have shown that there is a latency gradient of contraction along the esophagus that appears to contribute to generation of the peristaltic waves. Short duration of electrical stimulation of the intrinsic nerves of the circular smooth muscle strip results in a contraction that occurs after the stimulus has ended also called the off response.

The secondary phase is activated by the distention of the lumen. This can occur with the left behind food after the primary wave has subsided or it can also happen with the reflux contents from the stomach. This secondary wave or secondary phase is not associated with deglutition or pharyngeal or upper sphincter stimulation. The longitudinal smooth muscle contraction effects peristalsis by shortening the esophagus and by longitudinal contraction which slide this over the bolus. The contraction of the longitudinal muscles is longer distally than proximally.

The lower esophageal sphincter extending from 2 to 5 centimeters above the junction with the stomach and has circular muscle functions as a sphincter. It normally remains tonically constructed with a pressure at this point of about 30 millimeter of mercury. This is in stark contrast to the mid portion of the esophagus between the upper and lower sphincters which would normally remain relaxed. The tonic constriction of the lower esophageal sphincter helps to prevent any reflux from the stomach into the esophagus. When stimulated, the lower esophageal sphincter can have a sustained tone lasting several minutes, and can persist even after the stimulus is long past.

The smooth muscle of the gastrointestinal tract shows two basic types of electrical waves-slow waves and spikes. The voltage of the resting membrane potential of the gastrointestinal smooth muscle can change to different levels and this can have an impact on the activity off the tract. They always occur rhythmically. This rhythm is the frequency of the slow waves in the smooth muscle. These are however not action potentials, these are usually slow, until rating changes in the resting membrane potential which stop these slow waves are characterized by 5 to 15 millivolts of intensity and their frequency ranges from 3 to 12 per minute. It is about 3 three waves per minute in the body of the stomach and as much as 12 per minute in duodenum, and then it ranges from eight or nine in the jejunum and ileum. The slow waves cause muscle contraction in the stomach, however in the rest of the part of the gastrointestinal tract they mainly control the appearance of intermittent spike potentials, and these spike potentials in turn actually cause most of the muscle contractions. The frequency of slow waves drops to about 5 in colon before dramatically increasing to more than 15 per minute in the rectum.

Spike potentials are true action potentials and they happen because of the resting membrane potential of the gastrointestinal smooth muscle becomes more positive, 40 millivolts the normal resting membrane potential is somewhere between 52-60 millivolts. The frequency of the spike potential ranges between 1 to 10 spikes per second. Spike potential last about 10 to 40 times as long in the gastrointestinal muscle similar to the action potentials in the nerves.

The slow waves are termed the basic electric rhythm of the stomach, this is well characterized in the distal portion of the stomach. The fundus, proximal third of the stomach exhibits a sustained and non-phasic electrical activity. The gastric slow waves are generated by an area in mid corpus of the stomach along the greater curvature. They only move distally and are thus important in the emptying of the stomach. These waves travel myogenically and are not dependent on specialized nerve fibers such as the heart. The electrogastrogram can depict these changes from transcutaneous recordings, however, direct mucosal and serosal probes give a more accurate recording.

Embodiments of the device can initiate swallowing independently of the stimulation of the stomach. The device can stimulate and maintain stimulation of the lower esophageal sphincter to maintain its tone and prevent reflux. The series of esophageal electrodes/resonators act as sensors to sense ECG signals and any other biological electrical signal, amplifying it to prevent any inadvertent electrical stimulation that can interfere with cardiac activity or diaphragmatic activity. The electrodes which also act as an inductively coupled regimen can detect the gastric lumen, its contents, the direction of its flow and its response to the stimulation. Gastric stimulation is done on the basis of the signal information from the #9 and resultant effects are simultaneously measured in the duodenum, jejunum and pylorus creating an auto feedback loop. Jejunal stimulation effects and promoted gastric emptying, hence the jejunal feedback loop is created based on the electrode/resonators with inductive parameters to create effective bio-stimulation. Similar applications use stimulation in reverse order when one compares the upper, mid and lower rectum to treat constipation. The series of electrodes and resonators help to titrate the electrical signal, frequency, amplitude, and duration needed for effective defecation. The information and position of the electrodes/resonators can be accurately determined by a surface coil which can detect the EMF signal from various signals emitted. This configuration can also create a topographic map of organs the electrodes/resonators are near or within intraluminal.

Stimulation of any intraluminal organ can thus be customized based on application for example: intravesical (inside urinary bladder by mounted on a urinary catheter), within a ureter (on a ureteric catheter), external ear canal, external nasal passage, oral and pharyngeal cavities, thus it can be used for any cavity or organ with a lumen. More specifically, examples of application of use include:

Stimulating swallowing reflex by stimulating the posterior pharyngeal wall and above upper esophageal sphincter by stimulating the recurrent laryngeal nerve and glossopharyngeal nerve.

Feeding tube use for restoring GI peristalsis.

Intravesical use for acute urinary retention and to influence/increase urine output.

Intra-vaginal use to improve bladder neck tone, to correct urinary incontinence.

Endotracheal tube use for treating swallowing reflex dysfunction and to check nerve integrity.

Rectal tube use for treating constipation and to stimulate defecation.

External ear use for treating tinnitus.

Oral, pharyngeal use for treatment of dysphagia.

Post-operative use via nasogastric tube to stimulate appetite and food intake where the stimulating coils are placed at intervals to stimulate stomach and duodenal, jejunal contractions/stimulation. Gastric reflux can cause VAP and aspiration pneumonia, thus embodiments of the invention can be used to prevent this.

Post-operative use via nasogastric tube/endotracheal tube/tracheostomy tube to prevent aspiration. Paralysis weakness of the posterior pharyngeal sphincter and incomplete emptying or GI tract is responsible for majority of patients getting aspiration pneumonia, by periodically stimulating the emptying and contractions, aspiration can be avoided and prevented.

Post-operative use for periodic bowel emptying to prevent paralytic ileus and bowel impaction with fees. As a major cause of prolonged illness, narcotics use post operatively and prolonged recumbency can lead to slower bowel clearance which can lead to constipation and bowel organism translocation, this can be prevented by pacing stimulator coils within a rectal tube to stimulate bowel evacuation.

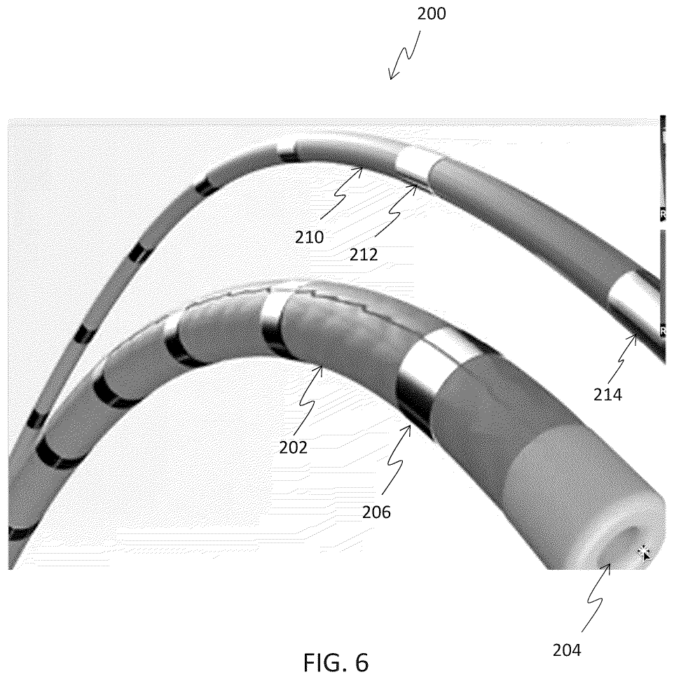

In one embodiment, with reference now to FIG. 6, a stimulation sheath 202 and internal body 210 that can be provided separately or as a kit 200 for use with an embodiment of the stimulation device (e.g. the described above in FIGS. 1A and 1B). The stimulation sheath 202 can include a set of stimulation elements 206 having a different alignment to stimulate a different or more focused part of the patient anatomy. For example, the stimulation sheath 202 can be configured to stimulate the lower esophageal sphincter only, instead of the entire length between the upper esophagus to the duodenum. The stimulation sheath 202 can be used for example instead of the stimulation device, or it can be loaded over the stimulation device then retracted once it's no longer needed, leaving the stimulation device in place. An internal body 210 can be included with additional stimulation elements 212 and one or more sensors 214, such as a pH sensor at the tip that sets at or protrudes from a distal opening 204 of the stimulation sheath 202. In one embodiment, the stimulator sheath 202 can be withdrawn partially while the internal body 210 remains stationary, allowing the medical professional the ability to elongate or shorten the stimulus area as needed. The stimulation sheath 202 and internal body 210 are elongate and flexible, and can include a plurality of resonator coils, a cathode connected to each of the plurality of resonator coils by a conductive lead at a cathode connection point, and multiple conductive leads for anodes each connected to and corresponding with one of the resonator coils at an anode connection point. The stimulation elements can be surface mount device (SMD) capacitors that are connected to and correspond with one of the resonator coils. A control unit similar to previous embodiments can be included. The controller can be programmed to selectively generate a sequence of stimulation signals for the stimulation elements based on detecting a predetermined threshold sequence of physiological feedback from the resonator coils.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

Animals were acclimatized in a central animal facility for 2 weeks before the experiment. All procedures were performed under aseptic conditions and anesthesia was achieved using a combination of ketamine and xylazine. Animals received humane care in compliance with the “Guide for Care and use of Laboratory Animals”, published by the National Research Council (National Academy Press, 1996).

### Surgical Model:

Various reflux was induced in 10-week-old, male Sprague Dawley rats, (200-250 gms, Harlan, Indianapolis, IN). The animals were allowed two weeks to acclimatize, and were housed at temperature of 20-22° C., humidity of 70%- and 12-hour alternating light-dark cycle. The rats were fasted overnight but allowed water ad-libitum till the surgery. Briefly, the esophagus was mobilized preserving the vagus nerves. The following models that were created to increase the severity of reflux of gastric contents:
(1) Laparotomy and measurement of electrical activity of esophagus, stomach, duodenum and jejunum-terminal experiment
(2) Cardio myotomy (surgical division of lower esophageal sphincter LES)
(3) Cardio-myotomy and pylorotomy
(4) Duodenal-esophageal anastomosis above LES
(5) Esophago-jejunal anastomosis

Once the animals awoke, they were allowed water ad-libitum. Feeding recommenced the following day. Animals received appropriate analgesia during the peri-operative and postoperative period.

Large animal preparation included, Yorkshire pigs which were anesthetized and intubated. A 110 cm long tube was then inserted via esophagus, then advanced to the stomach, duodenum, and jejunum to measure electrical activity within the structures and to provide electrical stimuli.

### Results:

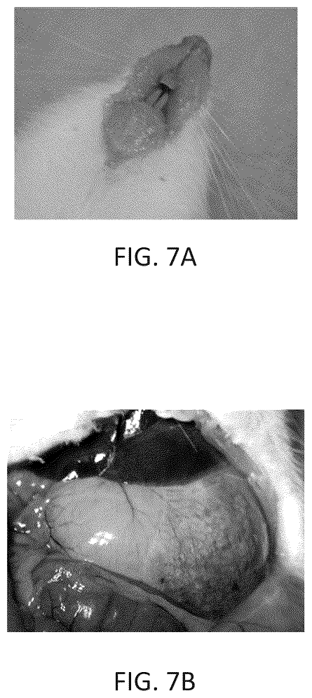

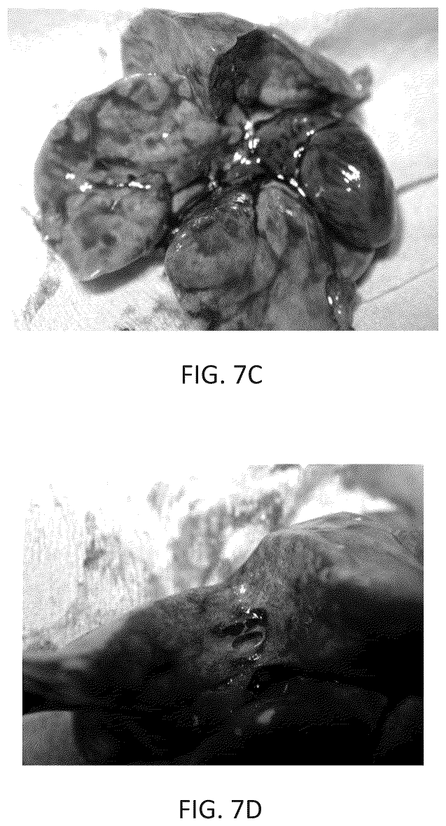

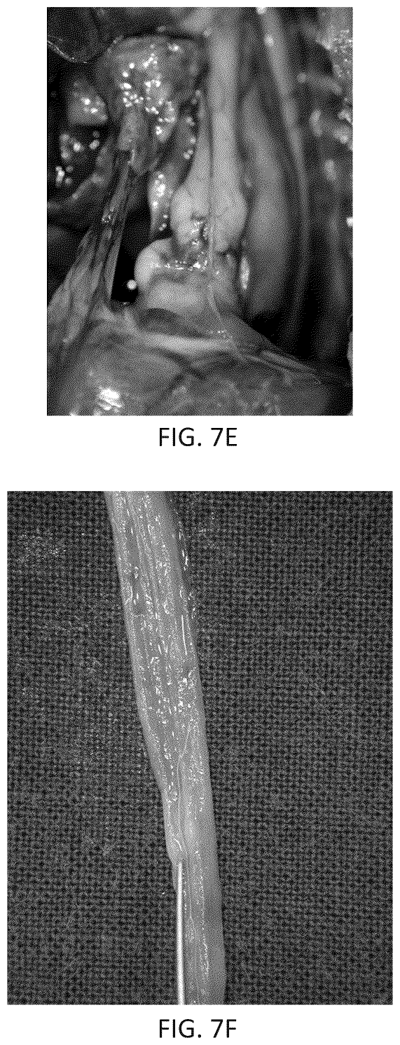

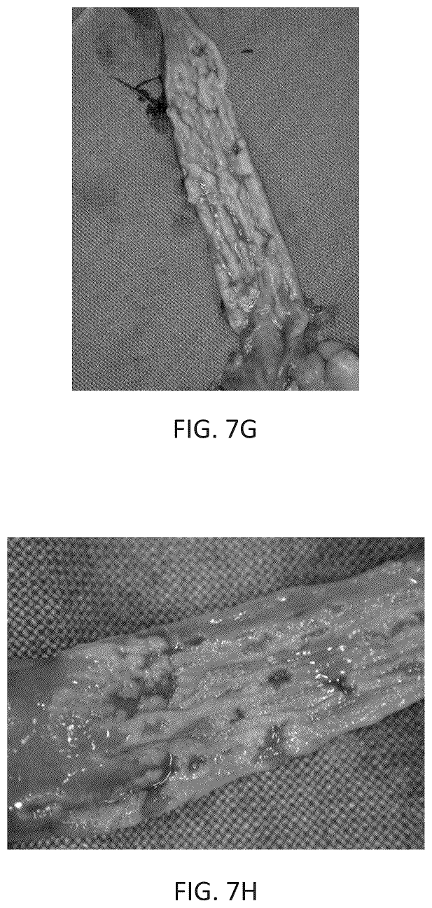

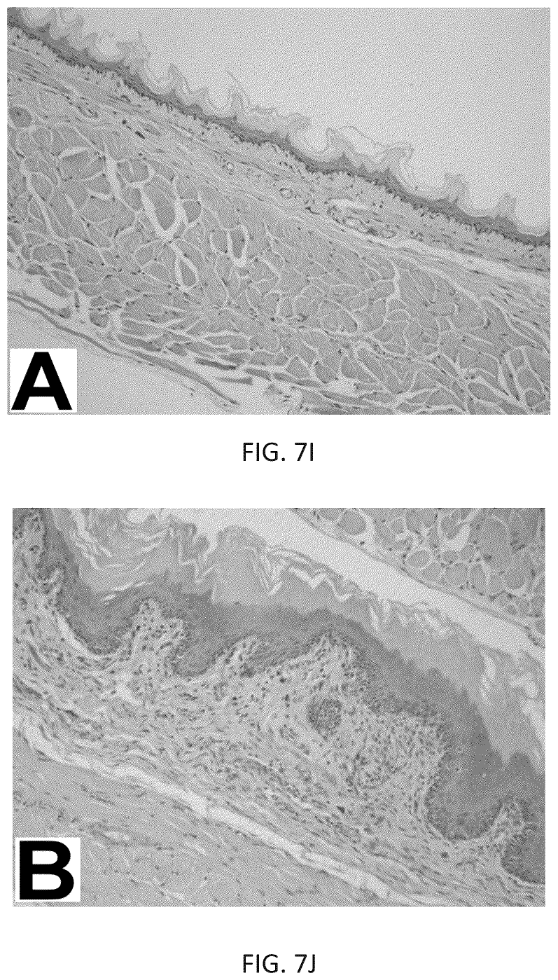

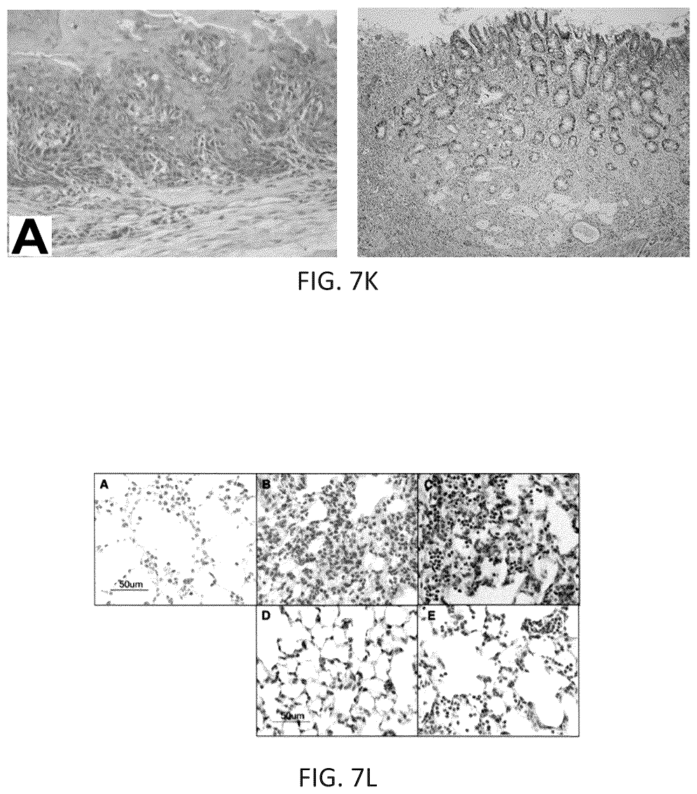

Gross appearance of reflux leading to increased oral secretions soiling the mouth and nasal area is shown in the image of FIG. 7A. Gastric distention with retained gastric contents is shown in the image of FIG. 7B. The gross appearance of aspiration pneumonia in lungs with increased weight, edema and infection is shown in FIG. 7C. 30% died within a week due to pneumonia, almost 100% showed evidence of aspiration pneumonia at autopsy in 2 weeks duration (untreated arm). Micro abscesses within the lung parenchyma are shown in FIG. 7D. Up to 15% of total or 50% of those died of pneumonia had visible abscesses. The gross appearance of esophagus with a distended lower portion is shown in FIG. 7E. A normal esophageal lumen appearance is shown in FIG. 7F. Reflux injury of the esophagus (gross appearance) is shown in FIGS. 6G and 6H. Normal esophagus (microscopic section) is shown in FIG. 7I, mild reflux injury is shown in FIG. 7J, and severe esophageal injury due to reflux is shown in FIG. 7K. Lung damage from reflux is shown in FIG. 7L (A: normal lung parenchyma control arm with no intervention). 100% showed B&C changes at 2 weeks on autopsy (30% died in untreated arm). All animals that died (30%) had all the severe changes of lung damage. Treated arm is shown in D&E with improved survival, only 2% mortality and improved histological evidence of minor reflux injury).

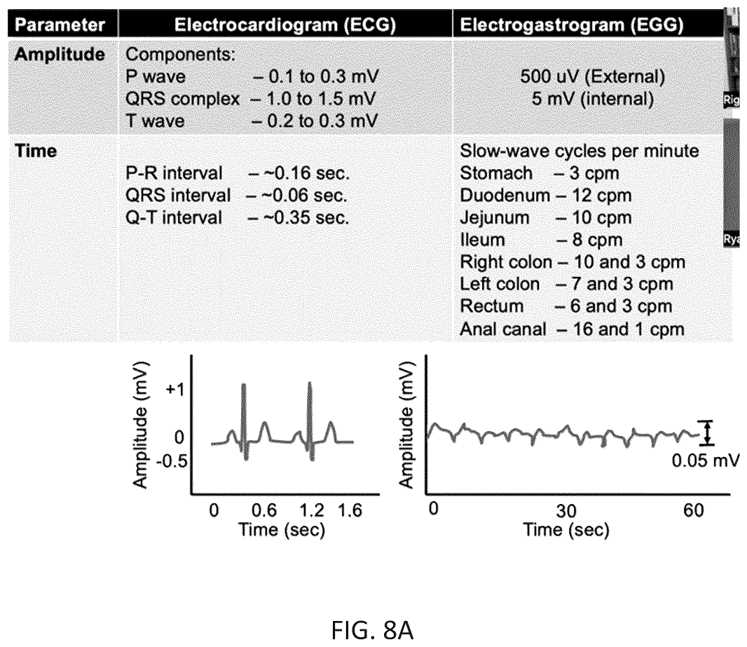

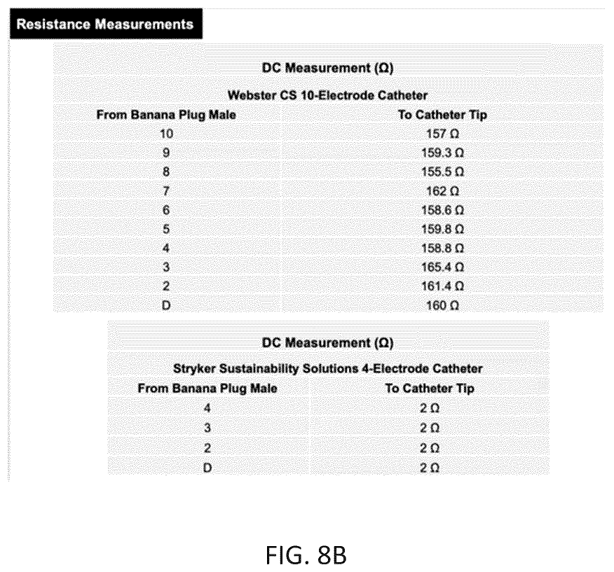

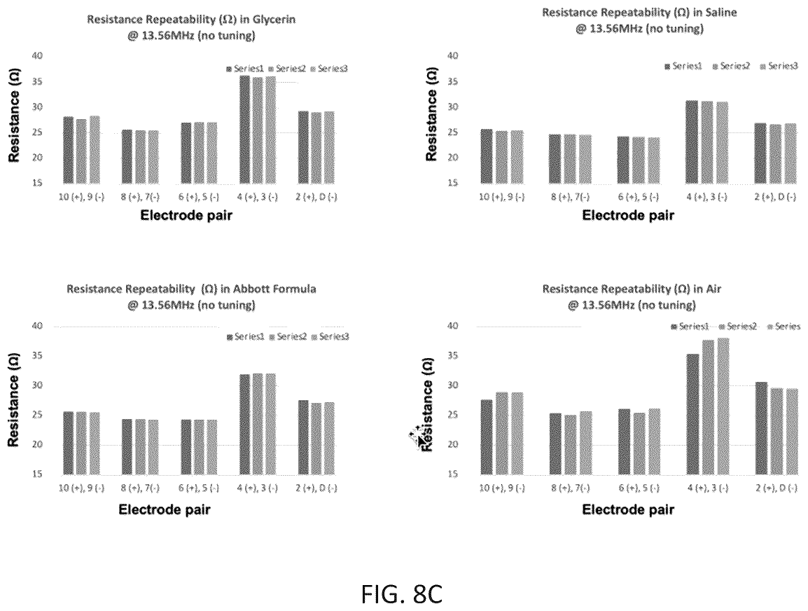

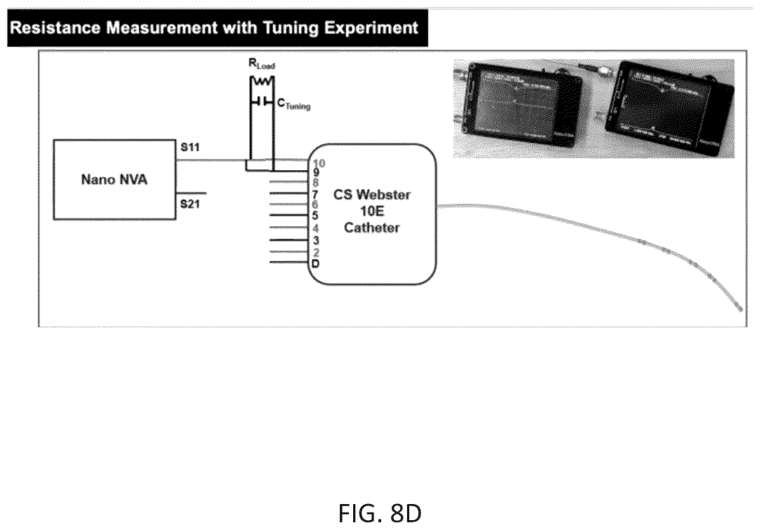

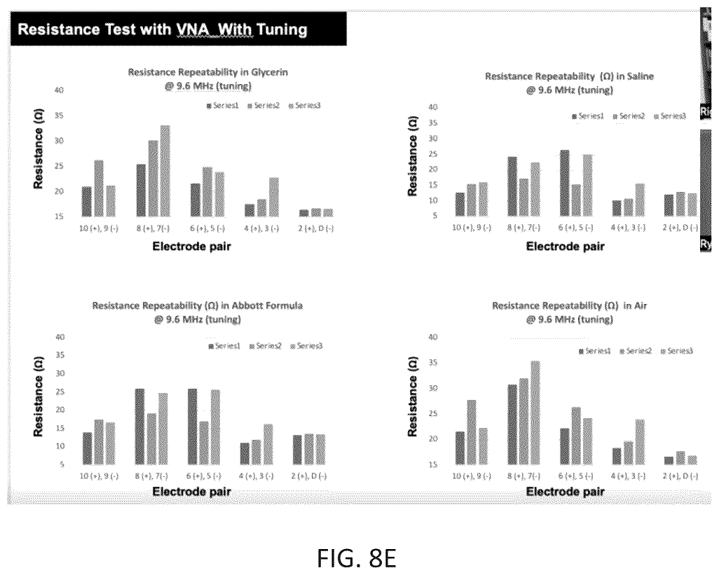

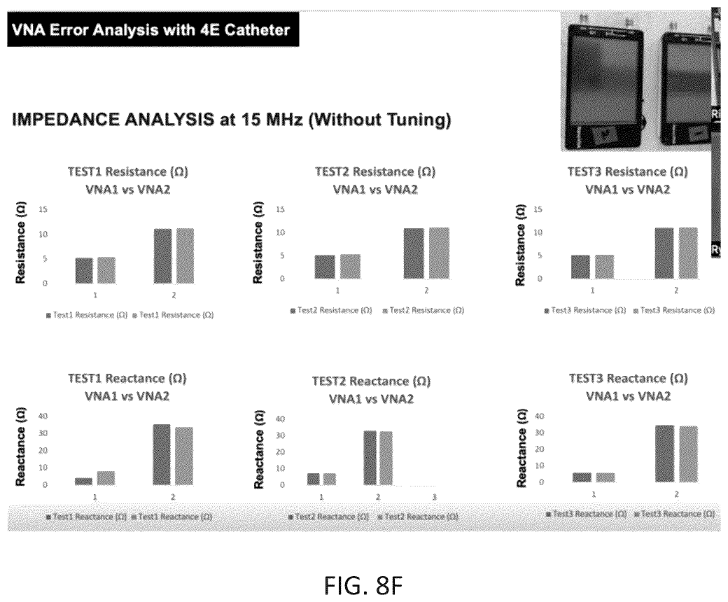

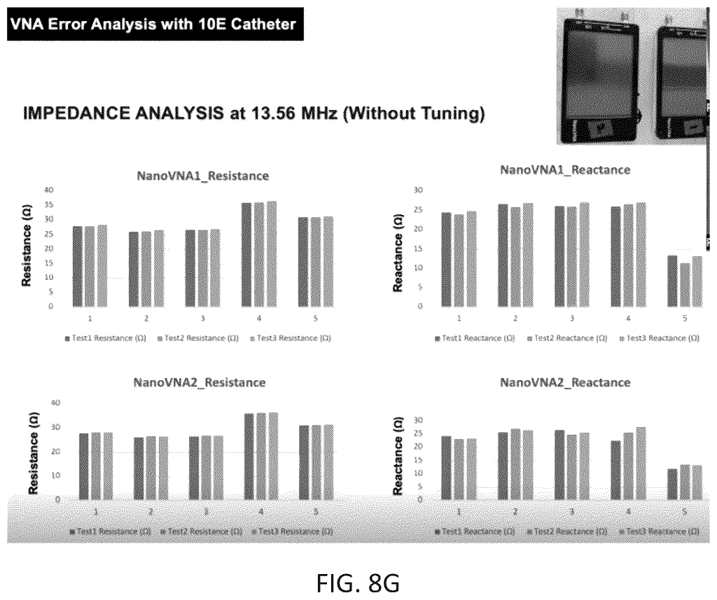

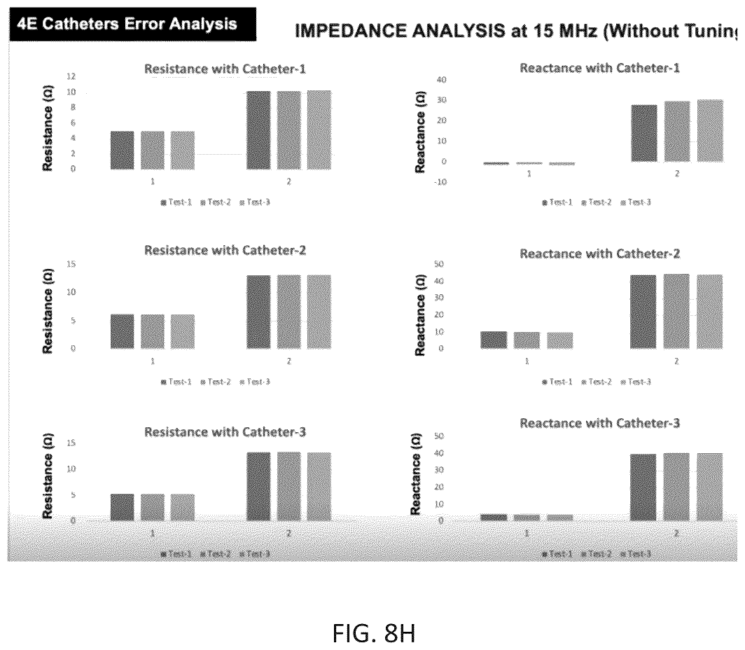

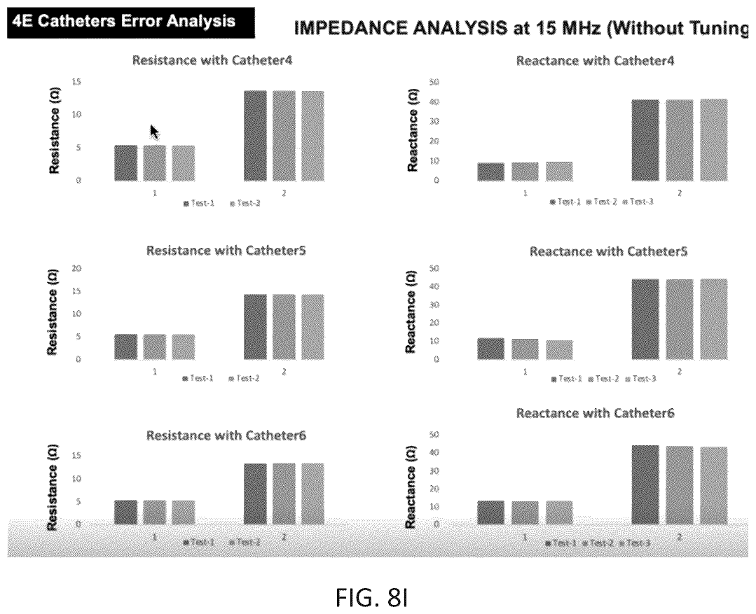

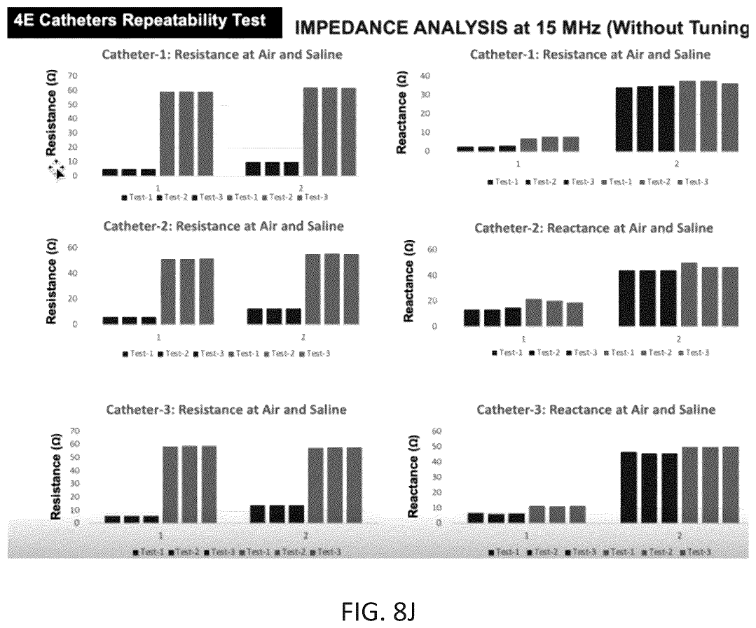

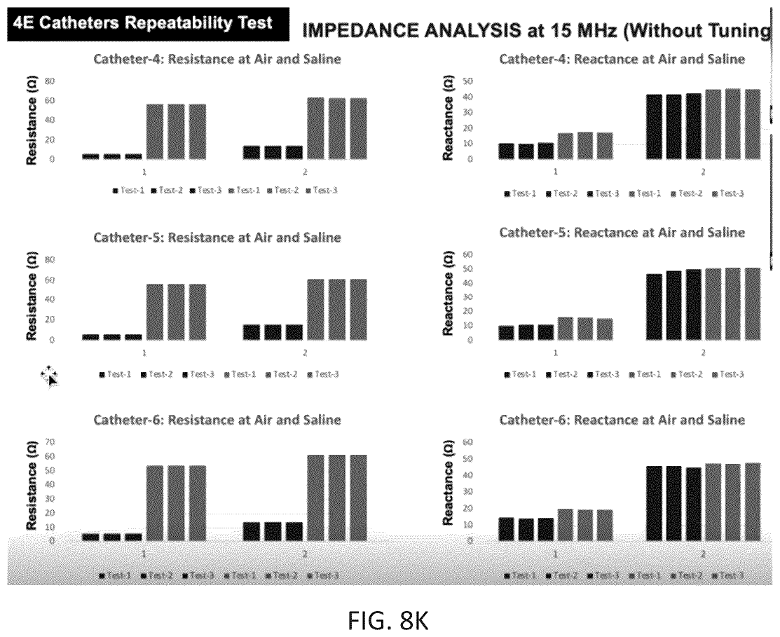

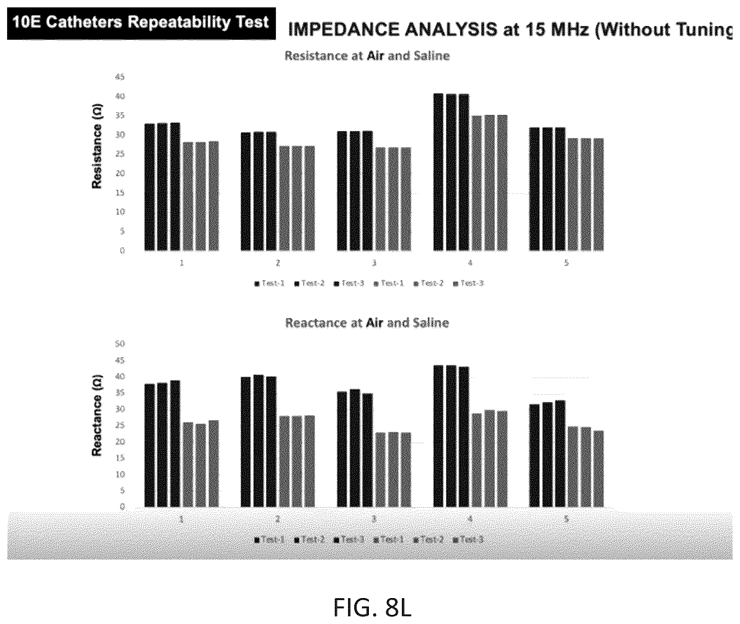

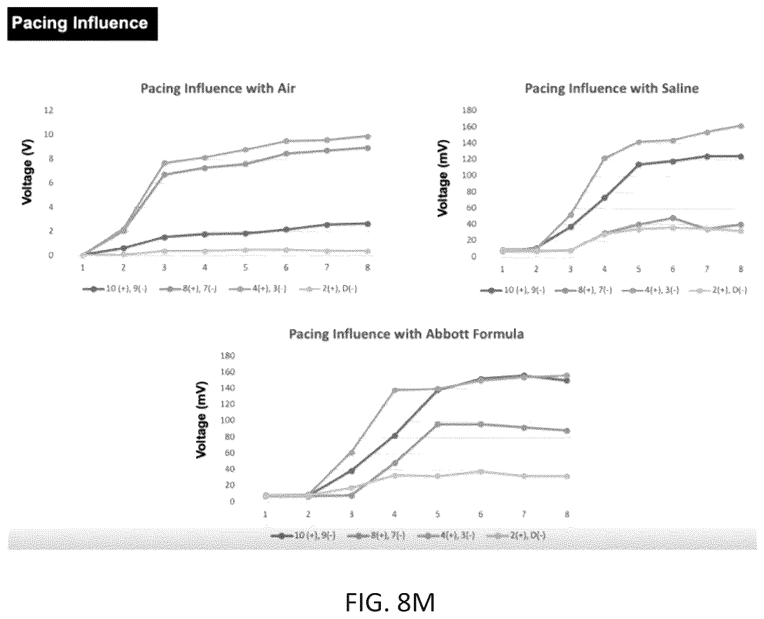

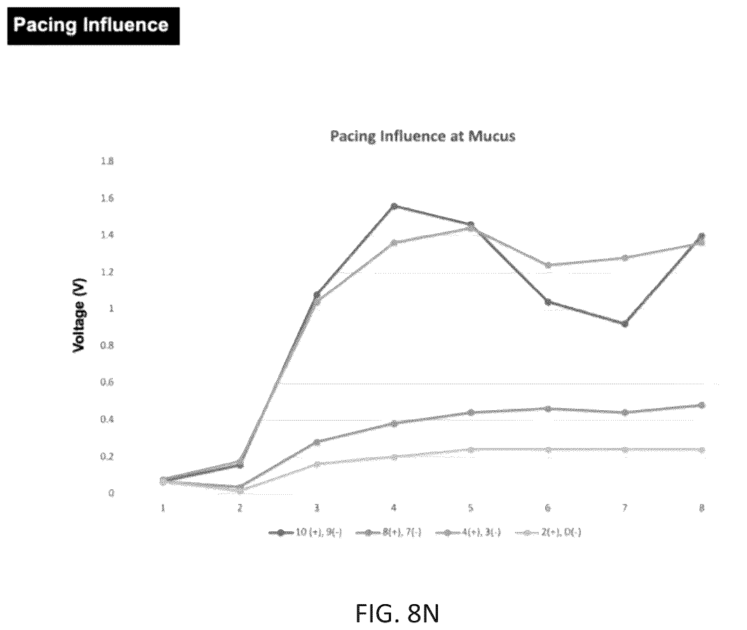

With reference now to FIG. 8A, the diagram according to one embodiment shows how the electrical activity of the heart compares to the electrical activity of the stomach. As shown, the signal generated in the stomach and esophagus is a very weak signal compared to the signal measured from the heart. The time row shows interval data, which in the heart is very short intervals and in the stomach and esophagus is much longer intervals. The pacing stimulus in the stomach and esophagus is therefore going to be vastly and less frequent compared to conventional ECG pacing devices and modalities.

With reference now to FIG. 8B, resistance measurements from a stimulation device having 10 electrodes are shown in the chart according to one embodiment. The number of electrodes in various embodiments may vary from 2 to 10 to 20 electrodes in certain examples.

With reference now to FIG. 8C experimental results are shown that demonstrate resistance is different in different medium. The first one is glycerin, the second one is saline, the third one is a feeding formula, and the last one is air. The X axis shows there are a different number of electrodes in series, and the number of times the experiments are conducted. There is a perceptible change between for example glycerin and saline, and for example glycerin and air. Based on the resistance measurement from the media, the system can predict which media the device is in. So for example, if it is air, it will be different. If it is water saline, it's detectable, etc. Accordingly, the system can differentiate between media. The system can vary the RF signal used to detect. So for example, as a particular frequency of electrical signal is selected, the system can increase or decrease the sensitivity of the media of interest.

With reference now to FIG. 8D, in one embodiment, an experimental setup is shown to demonstrate varying the RF signal, so for example, a different frequency of electrical signal can be implemented to increase or decrease sensitivity of the media of interest. The NanoVNA of channel-1 was connected to the CS catheter electrode pair (10-9 or 8-7 or 6-5 or 4-3 or 2-D . . . ) via RF coaxial cable as shown. In a pair of electrodes, one electrode was connected to the positive terminal of RF coaxial cable and the other electrode to the negative terminal. The reflection coefficient graph was displayed on the NanoVNA screen between 1 M and 20 MHz frequency span. A 40 nF external capacitance was used and achieved resonant frequency of 9.6 HMz. As shown in the above figure, the tuning circuit was between the RF coaxial cable and CS catheter banana male plug. Then, resistance and reactance graphs were displayed on the NanoVNA screen and analyzed impedance values for air, water, saline, feeding formula, and glycerin. The impedance formula is Z=Resistance (R)+Reactance (X).

With reference now to FIG. 8E, in one embodiment, it is possible to amplify the signal by spatially tuning two adjacent electrodes. The system can resonate two adjacent electrodes at the same frequency for better and enhanced sensitivity of detection (e.g. by selecting a particular RF signal band that matches resonant frequency).

With reference now to FIGS. 8F-8I, in one embodiment, experimental data is shown based on different types of catheters. These experiments demonstrate that embodiments of the invention can be adapted effectively with various catheter geometries. Specially fabricated tubes with four electrodes, ten electrodes, and twenty electrodes were used in testing. At 15 megahertz, the output is shown as very consistent and uniform. In the vector network analysis, no observer error is detected and results are repeatable. Looking at both resistance and impedance of the electrical current, results are consistent.

With reference now to FIGS. 8J-8L, in one embodiment, the experimental data looks at resistance for example in catheter 1 the blue (left three bars) is air and the brown (right three bars) is saline. The results clearly show the difference in resistance. Looking for example at catheter 4, catheter 5, catheter 6, the outcomes are very consistent across all catheters. The method described herein is highly repeatable. Three tests each for resistance level were conducted on a ten-electrode catheter. The repeatability demonstrated here is important, since in practice, medical professionals have to conduct repeated measurements that all need to be reliable. As shown by the data, there is no drift. No drift is important since it would otherwise show an electrical signal either losing signal or becoming erratic. The advantage here is that the data shows the embodiments demonstrate reproducible measurements with no drift, and high predictability across different sizes and environments.

With reference now to FIGS. 8M and 8N, in one embodiment, pacing data in reference to pacing the gastrointestinal tract is shown. A tiny electrical stimulus is applied to an electrode pair and the reflected waves in other electrodes can be measured. Looking at the Y axis (voltage), a large difference is observed when comparing for example air and salient measurements. Accordingly, the reflected wave will be very different when for example the device is in is air vs. saline, providing an additional method for confirming what is in the food pipe. Note that saline generally represents the reading detected for what is normally in a patient's stomach. This demonstrated that the reflected waves and be observed and relied upon for detecting different media and determining what type of content is in the food pipe.

Accordingly, the concept of putting a tube with the obturator within it has several advantages. That obturator can be disposed at the tip, it can have a conventional glass pH electrode, or it can also have optical fluorescence and or pressure transducer. In conventional methods, when you put the feeding tube, one of the biggest mistakes involves advancing it somewhere it's not supposed to go, such as an trachea, bronchus or even via lungs, blind passage can also go towards the brain upwards than downwards in the esophagus which is dangerous. This eliminates that need because by providing a roadmap to the stomach under vision (by rod lense, flexible optical fiber based, CMOS based camera system), or by detected pH levels, or by detecting optical fluorescence or by detecting pressure differential. Now, once you know you're in the stomach, it's safe to manipulate the device as needed. This safety feature ensures advancement into the stomach instead of e.g. the lungs or any other organs. In one embodiment, the device includes embedded pressure sensors on an external aspect of the hollow longitudinal tube configured to detect pressure at different levels of the esophagus, stomach, intestine or urinary bladder corresponding to a site of use. In one embodiment, the device includes an obturator within the outer sheath, where the obturator has a distal end viewing camera. In one embodiment, the device includes an obturator tip can having a pH sensor, optical fluorescence sensor and a pressure sensor, wherein the sheath is configured to be a carrier for a tube like structure within its lumen after the removal of the obturator.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A stimulation catheter comprising:
an elongate flexible member having a proximal end, a distal end, and a plurality of resonator coils configured therebetween;
a cathode connected to each of the plurality of resonator coils;
a plurality of anodes each connected to and corresponding with one of the plurality of resonator coils;
a plurality of stimulation elements each connected to and corresponding with one of the plurality of resonator coils; and
a control unit coupled to the cathode and the plurality of anodes, wherein the control unit is configured to generate a stimulation signal based on a plurality of feedback signals generated from the plurality of resonator coils.

2. The stimulation catheter of claim 1, wherein the control unit comprises a controller communicatively coupled to each of the plurality of anodes and each of the plurality of stimulation elements.

3. The stimulation catheter of claim 2, wherein the controller is configured to generate a sequence of stimulation signals for the plurality of stimulation elements.

4. The stimulation catheter of claim 3, wherein the sequence is based on detecting a predetermined threshold sequence of physiological feedback from the two or more of the plurality of resonator coils.

5. The stimulation catheter of claim 3, wherein the sequence activates the plurality of stimulation elements in an order moving in a distal direction.

6. The stimulation catheter of claim 3, wherein the sequence activates the plurality of stimulation elements in an order moving in a proximal direction.

7. The stimulation catheter of claim 3, wherein the sequence activates combinations of two or more of the plurality of stimulation elements at different times.

8. The stimulation catheter of claim 1, wherein the control unit comprises a multiplexer connected to each of the plurality of anodes.

9. The stimulation catheter of claim 1, wherein the plurality of feedback signals comprises at least one of a resistance, admittance, impedance, reactance, susceptance, capacitance, or conductance between inductively coupled electrode measurement signal.

10. The stimulation catheter of claim 1, wherein at least one of the plurality of feedback signals comprises a zero-value measurement.

11. The stimulation catheter of claim 1, wherein the plurality of resonator coils are spaced apart by a uniform distance.

12. The stimulation catheter of claim 1, wherein the plurality of resonator coils are spaced apart by different distances.

13. The stimulation catheter of claim 1, wherein the plurality of resonator coils are configured on an outer surface of the elongate flexible member.

14. The stimulation catheter of claim 13, wherein each of the plurality of resonator coils wraps around the outer surface at least one time in a spiral formation.

15. The stimulation catheter of claim 13, wherein each of the plurality of resonator coils wraps around an outer surface at least two times time in a spiral formation.

16. The stimulation catheter of claim 1, wherein the elongate flexible member comprises a lumen extending from the proximal end to a distal tip.

17. The stimulation catheter of claim 1, wherein the elongate flexible member comprises a medical grade polymer.

18. The stimulation catheter of claim 17, wherein the medical grade polymer is polyamide.

19. The stimulation catheter of claim 1, wherein at least one of the plurality of stimulation elements is a capacitor.

20. The stimulation catheter of claim 19, wherein the capacitor is a surface mount device capacitor configured on a surface of at least one of the plurality of resonator coils.

21. A stimulation catheter system comprising:
the catheter of claim 1; and
a elongate flexible sheath comprising a sheath lumen connected to a distal sheath opening, and plurality of sheath stimulation elements surrounding the sheath lumen.

22. A stimulation catheter system comprising:
the catheter of claim 1; and
an elongate flexible internal body having a distal tip sensor.

23. The stimulation catheter system of claim 22, wherein the distal tip sensor is a pH sensor.

24. The stimulation catheter system of claim 22, wherein the elongate flexible internal body comprises a plurality of body stimulation elements.

25. The stimulation catheter of claim 1, wherein the control unit is configured to differentiate between media based on a detected resistance.

26. The stimulation catheter of claim 1, wherein the control unit is configured to amplify detection by spatially tuning adjacent electrodes.

27. The stimulation catheter of claim 1 further comprising:
embedded pressure sensors on an external aspect of the hollow longitudinal tube configured to detect pressure at different levels of the esophagus, stomach, intestine or urinary bladder corresponding to a site of use.

28. The stimulation catheter of claim 1 further comprising:
an obturator within the outer sheath, where the obturator has a distal end viewing camera.

29. The stimulation catheter of claim 28 further comprising an obturator tip can having a pH sensor, optical fluorescence sensor and a pressure sensor, wherein the sheath is configured to be a carrier for a tube like structure within its lumen after the removal of the obturator.
